SEQUENCE LISTING (9 sequences)

SEQ ID NO: 1 - protein, 317 aa, Artificial sequence
  FVIII
EDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNATTIQNVSSNNSLSNNLISTDNTSSEENNDSKNVSSNNSAPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVT

SEQ ID NO: 2 - protein, 571 aa, Artificial sequence
  FVIII
EDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVT

SEQ ID NO: 3 - DNA, 162 bp, Artificial sequence
  FVIII B domain
tctttcagtcaaaatgctactaccatacagaatgtctcttccaataacagcctctcaaacaacctcatctcaactgacaacacttcttctgaggagaacaatgacagtaagaatgtgtcctccaataattcagcgcccccagtattgaaacgccaccagagg

SEQ ID NO: 4 - DNA, 924 bp, Artificial sequence
  FVIII B domain
agcttttcccagaatagccgacatcctagcactcgccaaaaacagtttaatgcgacaactatccctgagaacgatattgagaaaactgatccctggtttgcacatcgcactcctatgccaaagatccaaaacgtgagcagctctgacctccttatgttgcttagacaatctcccacacctcatggactctcactttccgatctgcaggaggcgaagtatgaaaccttctcagacgacccatccccaggagccatagactcaaacaatagtctctcagaaatgacgcactttagacctcaactccatcacagtggggatatggtatttacccccgagagtggtctgcagcttaggcttaatgaaaaattgggaaccaccgctgcaacggaactcaaaaaactggacttcaaggtttctagcacgtcaaacaatcttatatcaaccataccatccgacaaccttgccgcaggaaccgataacacatcaagcctggggcctccatcaatgccggtgcactatgattcacagttggatactaccctcttcgggaagaaaagttcaccgctgactgaaagcggtggcccactgtctctgagtgaagagaataatgattctaaacttctcgagagcggcctcatgaatagtcaggagagttcttgggggaaaaatgttagcagtactgagagcggacggctcttcaaaggtaagcgggcacatgggcccgctcttctgactaaggataacgctttgttcaaagttagcatatcactcctgaaaactaacaagacctcaaataattctgcaacgaaccggaagacccatattgacggtccaagtttgctcatcgagaactccccgagtgtatggcagaacattcttgagagcgataccgagtttcccccggtactcaagaggcatcagcgg

SEQ ID NO: 5 - DNA, 4494 bp, Artificial sequence
  F8-BDD-N8
atgcagatcgaactgagcacctgcttcttcctgtgtctcctgagattctgctttagtgctaccagacggtattacctgggagccgtcgagctgagttgggattacatgcagtccgacctcggagaactgcctgtggatgcacgctttccaccaagagtgcctaagtcattcccattcaacacctcagtcgtgtataagaagactctgttcgtcgagtttactgatcacctgttcaatatcgctaaacctagaccaccctggatgggactgctgggtcctacaatccaggcagaggtctatgacactgtggtgattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagaatttcaggccagtaatataatgcactccatcaacggatatgtctttgactccttgcaactctcagtgtgtcttcacgaggtggcctattggtatattctcagcataggggcccagactgactttctgtctgtcttcttcagcggatatacttttaagcataagatggtttatgaggatacattgacattgttccctttcagtggggagaccgtctttatgtctatggaaaatcctgggctctggatactcggttgccacaatagtgacttccgaaatcgcggaatgacagctctgctgaaagtgtccagttgtgacaaaaacaccggggactattacgaagacagctatgaagatataagtgcatatttgctcagcaagaacaatgcgattgagccaaggtctttcagtcaaaatgctactaccatacagaatgtctcttccaataacagcctctcaaacaacctcatctcaactgacaacacttcttctgaggagaacaatgacagtaagaatgtgtcctccaataattcagcgcccccagtattgaaacgccaccagagggagatcaccagaaccacactgcagtcagaccaagaggaaattgactatgatgacacaatcagtgtcgaaatgaaaaaagaagactttgatatttacgatgaggatgaaaatcagtcaccaagatcctttcaaaaaaaaacccgacattattttatagcagccgtcgaacggttgtgggattatggcatgagctcaagtccacatgtactgagaaatagggcgcagtcaggaagcgtaccccagtttaagaaggttgtattccaagaattcacagacggtagctttacccagccgctttatcgaggagagttgaatgagcaccttggtttgctgggaccgtacatccgcgcagaagtcgaagacaatataatggtcacctttcggaaccaagcctccaggccatacagtttctacagttctctgatctcatacgaggaagatcagaggcaaggagcagaaccaaggaagaacttcgtgaaaccaaacgagacaaagacctatttctggaaagttcagcatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 6 - DNA, 5256 bp, Artificial sequence
  F8-BDD-299
atgcagatcgaactgagcacctgcttcttcctgtgtctcctgagattctgctttagtgctaccagacggtattacctgggagccgtcgagctgagttgggattacatgcagtccgacctcggagaactgcctgtggatgcacgctttccaccaagagtgcctaagtcattcccattcaacacctcagtcgtgtataagaagactctgttcgtcgagtttactgatcacctgttcaatatcgctaaacctagaccaccctggatgggactgctgggtcctacaatccaggcagaggtctatgacactgtggtgattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccggagtttcaggcatccaatatcatgcattctataaacgggtatgtatttgattctttgcagttgagtgtgtgtctgcatgaggttgcctactggtacattctgtctataggggcgcagacggatttcctttcagtgttcttcagcgggtatacatttaaacataagatggtatatgaggacaccttgacattgtttccattttccggcgaaaccgtattcatgtcaatggagaacccagggttgtggatactcggttgccataatagtgacttcagaaaccgagggatgacggcccttctcaaagtaagttcatgtgataagaataccggtgattactacgaagatagctatgaggatattagcgcctacttgcttagcaagaataacgctattgaacctaggagcttttcccagaatagccgacatcctagcactcgccaaaaacagtttaatgcgacaactatccctgagaacgatattgagaaaactgatccctggtttgcacatcgcactcctatgccaaagatccaaaacgtgagcagctctgacctccttatgttgcttagacaatctcccacacctcatggactctcactttccgatctgcaggaggcgaagtatgaaaccttctcagacgacccatccccaggagccatagactcaaacaatagtctctcagaaatgacgcactttagacctcaactccatcacagtggggatatggtatttacccccgagagtggtctgcagcttaggcttaatgaaaaattgggaaccaccgctgcaacggaactcaaaaaactggacttcaaggtttctagcacgtcaaacaatcttatatcaaccataccatccgacaaccttgccgcaggaaccgataacacatcaagcctggggcctccatcaatgccggtgcactatgattcacagttggatactaccctcttcgggaagaaaagttcaccgctgactgaaagcggtggcccactgtctctgagtgaagagaataatgattctaaacttctcgagagcggcctcatgaatagtcaggagagttcttgggggaaaaatgttagcagtactgagagcggacggctcttcaaaggtaagcgggcacatgggcccgctcttctgactaaggataacgctttgttcaaagttagcatatcactcctgaaaactaacaagacctcaaataattctgcaacgaaccggaagacccatattgacggtccaagtttgctcatcgagaactccccgagtgtatggcagaacattcttgagagcgataccgagtttcccccggtactcaagaggcatcagcgggagattacgcgaaccacactccagtccgatcaggaagaaattgattatgacgatactatcagtgtagagatgaaaaaagaagactttgacatctatgatgaggacgagaaccagtctccacgaagctttcagaaaaaaacaaggcactattttatcgccgctgttgaacggctgtgggactacggtatgtcctcttcaccccacgtgctgcggaaccgggcccagtcaggctcagtaccccaattcaagaaggtggtattccaggaatttaccgatggatctttcacgcaacctctttaccgaggtgagctgaacgaacatcttggccttctcggtccttatattagagcagaggtggaagacaatataatggtcacctttcggaaccaagcctccaggccatacagtttctacagttctctgatctcatacgaggaagatcagaggcaaggagcagaaccaaggaagaacttcgtgaaaccaaacgagacaaagacctatttctggaaagttcagcatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 7 - DNA, 4374 bp, Artificial sequence
  F8-BDD
atgcagatcgaactgagcacctgcttcttcctgtgtctcctgagattctgctttagtgctaccagacggtattacctgggagccgtcgagctgagttgggattacatgcagtccgacctcggagaactgcctgtggatgcacgctttccaccaagagtgcctaagtcattcccattcaacacctcagtcgtgtataagaagactctgttcgtcgagtttactgatcacctgttcaatatcgctaaacctagaccaccctggatgggactgctgggtcctacaatccaggcagaggtctatgacactgtggtgattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttttctcagaatcctcctgtcctcaaacgccatcaacgggagattacacggaccacactccaaagcgatcaggaggagatcgactatgacgataccatatctgtggaaatgaagaaagaggacttcgacatctacgacgaagatgagaaccaaagtccaagatccttccagaagaagactaggcactacttcatcgctgccgtggaacgcctctgggattacggaatgtccagttctccacatgtcctcaggaatagggcacagtctggctctgttccacagtttaagaaagttgtctttcaggagttcacagatggctcattcactcaaccactgtatagaggcgaactgaatgagcacctgggactgctgggtccctacatcagagccgaagtggaggataacattatggtcacctttcggaaccaagcctccaggccatacagtttctacagttctctgatctcatacgaggaagatcagaggcaaggagcagaaccaaggaagaacttcgtgaaaccaaacgagacaaagacctatttctggaaagttcagcatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 8 - protein, 54 aa, Artificial sequence
  B domain - N8
SFSQNATTIQNVSSNNSLSNNLISTDNTSSEENNDSKNVSSNNSAPPVLKRHQR

SEQ ID NO: 9 - protein, 308 aa, Artificial sequence
  B domain - 299
SFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFPPVLKRHQR

(19) United States
(12) Patent Application Publication
(10) Pub. No.: US 2024/0218048 A1
(43) Pub. Date: Jul. 4, 2024

(54) RECOMBINANT COAGULATION FACTOR VIII AND USE THEREOF

(71) Applicant: BEIJING MEIKANG GENO-IMMUNE BIOTECHNOLOGY CO., LTD., Beijing (CN)

(72) Inventors: Lung-Ji Chang, Beijing (CN); Jie Gong, Beijing (CN); Rui Zhang, Beijing (CN)

(21) Appl. No.: 18/557,147

(22) PCT Filed: Apr. 8, 2022
(86) PCT No.: PCT/CN2022/085853

§ 371 Date: Oct. 25, 2023

(30) Foreign Application Priority Data
Apr. 26, 2021 (CN) 202110455812.5
Jul. 6, 2021 (CN) 202110762308.X

(51) Int. Cl. C07K 14/755; A61K 38/00; A61P 7/04; C12N 15/86

(52) U.S. Cl. CPC C07K 14/755; A61P 7/04; C12N 15/86; A61K 38/00; C12N 2740/15043; C12N 2840/44

(57) ABSTRACT

Provided are recombinant coagulation factor VIII and application use thereof. The recombinant coagulation factor VIII includes more than 80% of an amino acid sequence as shown in SEQ ID NO: 1 or SEQ ID NO: 2. Through gene modification of the B domain, the recombinant coagulation factor VIII is rich in glycosylation sites, has a good coagulation function, is easily secreted outside a cell and easily interacts with a cofactor such as thrombin (Ella). In addition, the recombinant coagulation factor VIII has a weak ability to induce an antibody response and can effectively ensure a treatment effect, reduce a risk of immune rejection and lower a treatment cost.

### CROSS-REFERENCE TO RELATED APPLICATION(S)

This application claims priority to Chinese Patent Application No. CN 202110455812.5 filed on Apr. 26, 2021, the disclosure of which is incorporated herein by reference in its entirety.

### TECHNICAL FIELD

The present application belongs to the field of biotechnology and relates to recombinant coagulation factor VIII and a use thereof.

### BACKGROUND

Hemophilia A (HA), also known as a hereditary anti-hemophilic globulin deficiency or an FVIII deficiency, is a coagulation disorder caused by a genetic deficiency in a coagulation factor VIII gene (FVIII gene or F8 gene). At present, the HA is mainly treated by protein replacement therapy (RPT) based on a plasma-derived coagulation factor or a recombinant protein exogenously cultured. However, PRT has limitations such as a short half-life, a high cost and a long treatment period.

Gene therapy refers to that a normal exogenous gene is introduced into target cells to correct or compensate for a defective gene and an abnormal gene for the purpose of treating a disease caused by the defective gene and the abnormal gene. At present, gene therapy is considered to be the most promising treatment for HA.

The analysis of a human F8 gene shows that an expression protein of the human F8 gene has distinct domains expressed as A1-A2-B-A3-C1-C2. The B domain is encoded by a very large exon and contains a highly conserved region consisting of asparagine (N) linked to oligosaccharides. Miao et al. pointed out that a partial deletion of the B domain retaining 226 amino acids at an N-terminal which contain six intact asparagine-linked glycosylation sites can increase the in vitro secretion of F8 by ten times (see Miao, H. Z., Sirachainan, N., Palmer, L., Kucab, P., Cunningham, M. A. et al. Bioengineering of coagulation factor VIII for improved secretion. Blood, 2004, 103(9), 3412-3419.). However, gene therapy using a B-domain-deleted F8 gene (F8-BDD) has problems such as low protein secretion and function, low transduction efficiency of an F8 viral vector and a response related to an antibody and an inhibitor formation (immune rejection).

In summary, recombinant coagulation factor VIII is provided, which has a good coagulation function and a weak in vivo induced response of an inhibitory antibody and is of great significance in the field of HA gene therapy.

### SUMMARY

The present application provides a recombinant coagulation factor VIII and a use thereof. The recombinant coagulation factor VIII is rich in glycosylation sites, has a good coagulation function and a weak response to an antibody, is easily secreted outside a cell, and can efficiently correct hemophilia A.

In a first aspect, the present application provides recombinant coagulation factor VIII, which includes more than 80% of an amino acid sequence as shown in SEQ ID NO: 1 or SEQ ID NO: 2.

The recombinant coagulation factor VIII of the present application is rich in sequences of glycosylation sites, has a good coagulation function, is easily secreted outside a cell and easily interacts with a cofactor such as thrombin (FIIa). In addition, the recombinant coagulation factor VIII has a weak response to induce an antibody and can effectively ensure a treatment effect, reduce a risk of immune rejection and lower a treatment cost.

SEQ ID NO: 1: (underlined is a synthetic B domain [N8] in the present application, and A2 and A3 domains are in front of and behind the B domain, respectively)

EDPEFQASNIMHSINGYVFDSLQLSVCLHEVAYWYILSIGAQTDFLSVFFSGYTFKHKM
VYEDTLTLFPFSGETVFMSMENPGLWILGCHNSDFRNRGMTALLKVSSCDKNTGDYYEDS
YEDISAYLLSKNNAIEPRSFSQNATTIQNVSSNNSLSNNLISTDNTSSEENNDSKNVSSNNSAP
PVLKRHQREITRTTLQSDQEEIDYDDTISVEMKKEDFDIYDEDENQSPRSFQKKTRHYFIAA
VERLWDYGMSSSPHVLRNRAQSGSVPQFKKVVFQEFTDGSFTQPLYRGELNEHLGLLGPYI
RAEVEDNIMVT.
SEQ ID NO: 2: (underlined is a synthetic B domain [299] in the present application,
and A2 and A3 domains are in front of and behind the B domain, respectively)
EDPEFQASNIMHSINGYVFDSLQLSVCLHEVAYWYILSIGAQTDFLSVFFSGYTFKHKM
VYEDTLTLFPFSGETVFMSMENPGLWILGCHNSDFRNRGMTALLKVSSCDKNTGDYYEDS
YEDISAYLLSKNNAIEPRSFSQNSRHPSTRQKQFNATTIPENDIEKTDPWFAHRTPMPKIQNV
SSSDLLMLLRQSPTPHGLSLSDLQEAKYETFSDDPSPGAIDSNNSLSEMTHFRPQLHHSGDM
VFTPESGLQLRLNEKLGTTAATELKKLDFKVSSTSNNLISTIPSDNLAAGTDNTSSLGPPSMP
VHYDSQLDTTLFGKKSSPLTESGGPLSLSEENNDSKLLESGLMNSQESSWGKNVSSTESGRL
FKGKRAHGPALLTKDNALFKVSISLLKTNKTSNNSATNRKTHIDGPSLLIENSPSVWQNILES
DTEFPPVLKRHQREITRTTLQSDQEEIDYDDTISVEMKKEDFDIYDEDENQSPRSFQKKTRH
YFIAAVERLWDYGMSSSPHVLRNRAQSGSVPQFKKVVFQEFTDGSFTQPLYRGELNEHLGL
LGPYIRAEVEDNIMVT.

In some embodiments of the present application, the recombinant coagulation factor VIII contains a B domain which includes an amino acid sequence as shown in SEQ ID NO: 8:

SEQ ID NO: 8: (B domain [N8])
SFSQNATTIQNVSSNNSLSNNLISTDNTSSEENNDSKNVSSNNSAPPVL
KRHQR.

In some embodiments of the present application, the recombinant coagulation factor VIII contains a B domain which includes an amino acid sequence as shown in SEQ ID NO: 9:

SEQ ID NO: 9: (B domain [299])
SFSQNSRHPSTRQKQFNATTIPENDIEKTDPWFAHRTPMPKIQNVSSSD
LLMLLRQSPTPHGLSLSDLQEAKYETFSDDPSPGAIDSNNSLSEMTHFR
PQLHHSGDMVFTPESGLQLRLNEKLGTTAATELKKLDFKVSSTSNNLIS
TIPSDNLAAGTDNTSSLGPPSMPVHYDSQLDTTLFGKKSSPLTESGGPL
SLSEENNDSKLLESGLMNSQESSWGKNVSSTESGRLFKGKRAHGPALLT
KDNALFKVSISLLKTNKTSNNSATNRKTHIDGPSLLIENSPSVWQNILE
SDTEFPPVLKRHQR.

In a second aspect, the present application provides a genetic sequence as shown in SEQ ID NO:3 and SEQ ID NO:4 encoding a B domain of recombinant coagulation factor VIII of the present application.

SEQ ID NO: 3:
tctttcagtcaaaatgctactaccatacagaatgtctcttccaataacagcctctcaaacaacctcatctcaactgacaacacttcttctgagg
agaacaatgacagtaagaatgtgtcctccaataattcagcgcccccagtattgaaacgccaccagagg.
SEQ ID NO: 4:
agcttttcccagaatagccgacatcctagcactcgccaaaaacagtttaatgcgacaactatccctgagaacgatattgagaaaactgatc
cctggtttgcacatcgcactcctatgccaaagatccaaaacgtgagcagctctgacctccttatgttgcttagacaatctcccacacctcatgga
ctctcactttccgatctgcaggaggcgaagtatgaaaccttctcagacgacccatccccaggagccatagactcaaacaatagtctctcagaaat
gacgcactttagacctcaactccatcacagtggggatatggtatttacccccgagagtggtctgcagcttaggcttaatgaaaaattgggaacca
ccgctgcaacggaactcaaaaaactggacttcaaggtttctagcacgtcaaacaatcttatatcaaccataccatccgacaaccttgccgcagga
accgataacacatcaagcctggggcctccatcaatgccggtgcactatgattcacagttggatactaccctcttcgggaagaaaagttcaccgct
gactgaaagcggtggcccactgtctctgagtgaagagaataatgattctaaacttctcgagagcggcctcatgaatagtcaggagagttcttggg
ggaaaaatgttagcagtactgagagcggacggctcttcaaaggtaagcgggcacatgggcccgctcttctgactaaggataacgctttgttcaaa
gttagcatatcactcctgaaaactaacaagacctcaaataattctgcaacgaaccggaagacccatattgacggtccaagtttgctcatcgagaa
ctccccgagtgtatggcagaacattcttgagagcgataccgagtttcccccggtactcaagaggcatcagcgg.

In some specific embodiments, the gene encoding the recombinant coagulation factor VIII includes a nucleic acid sequence as shown in SEQ ID NO: 5 or SEQ ID NO: 6.

SEQ ID NO: 5: (a nucleic acid sequence encoding a full-length F8-BDD-N8 protein)
atgcagatcgaactgagcacctgcttcttcctgtgtctcctgagattctgctttagtgctaccagacggtattacctgggagccgtcgagctg
agttgggattacatgcagtccgacctcggagaactgcctgtggatgcacgctttccaccaagagtgcctaagtcattcccattcaacacctcagtc
gtgtataagaagactctgttcgtcgagtttactgatcacctgttcaatatcgctaaacctagaccaccctggatgggactgctgggtcctacaatc
caggcagaggtctatgacactgtggtgattacacttaagaacatggcttcccatcctgtcagtcttcatgctgttggtgtatcctactggaaagct
tctgagggagctgaatatgatgatcagaccagtcaaagggagaaagaagatgataaagtcttccctggtggaagccatacatatgtctggcaggtc
ctgaaagagaatggtccaatggcctctgacccactgtgccttacctactcatatctttctcatgtggacctggtaaaagacttgaattcaggcctc
attggagccctactagtatgtagagaagggagtctggccaaggaaaagacacagaccttgcacaaatttatactactttttgctgtatttgatgaa
gggaaaagttggcactcagaaacaaagaactccttgatgcaggatagggatgctgcatctgctcgggcctggcctaaaatgcacacagtcaatggt
tatgtaaacaggtctctgccaggtctgattggatgccacaggaaatcagtctattggcatgtgattggaatgggcaccactcctgaagtgcactca
atattcctcgaaggtcacacatttcttgtgaggaaccatcgccaggcgtccttggaaatctcgccaataactttccttactgctcaaacactcttg
atggaccttggacagtttctactgttttgtcatatctcttcccaccaacatgatggcatggaagcttatgtcaaagtagacagctgtccagaggaa
ccccaactacgaatgaaaaataatgaagaagcggaagactatgatgatgatcttactgattctgaaatggatgtggtcaggtttgatgatgacaac
tctccttcctttatccaaattcgctcagttgccaagaagcatcctaaaacttgggtacattacattgctgctgaagaggaggactgggactatgct
cccttagtcctcgcccccgatgacagaagttataaaagtcaatatttgaacaatggccctcagcggattggtaggaagtacaaaaaagtccgattt
atggcatacacagatgaaacctttaagactcgtgaagctattcagcatgaatcaggaatcttgggacctttactttatggggaagttggagacaca
ctgttgattatatttaagaatcaagcaagcagaccatataacatctaccctcacggaatcactgatgtccgtcctttgtattcaaggagattacca
aaaggtgtaaaacatttgaaggattttccaattctgccaggagaaatattcaaatataaatggacagtgactgtagaagatgggccaactaaatca
gatcctcggtgcctgacccgctattactctagtttcgttaatatggagagagatctagcttcaggactcattggccctctcctcatctgctacaaa
gaatctgtagatcaaagaggaaaccagataatgtcagacaagaggaatgtcatcctgttttctgtatttgatgagaaccgaagctggtacctcaca
gagaatatacaacgctttctccccaatccagctggagtgcagcttgaggatccagaatttcaggccagtaatataatgcactccatcaacggatat
gtctttgactccttgcaactctcagtgtgtcttcacgaggtggcctattggtatattctcagcataggggcccagactgactttctgtctgtcttc
ttcagcggatatacttttaagcataagatggtttatgaggatacattgacattgttccctttcagtggggagaccgtctttatgtctatggaaaat
cctgggctctggatactcggttgccacaatagtgacttccgaaatcgcggaatgacagctctgctgaaagtgtccagttgtgacaaaaacaccggg
gactattacgaagacagctatgaagatataagtgcatatttgctcagcaagaacaatgcgattgagccaaggtctttcagtcaaaatgctactacc
atacagaatgtctcttccaataacagcctctcaaacaacctcatctcaactgacaacacttcttctgaggagaacaatgacagtaagaatgtgtcc
tccaataattcagcgcccccagtattgaaacgccaccagagggagatcaccagaaccacactgcagtcagaccaagaggaaattgactatgatgac
acaatcagtgtcgaaatgaaaaaagaagactttgatatttacgatgaggatgaaaatcagtcaccaagatcctttcaaaaaaaaacccgacattat
tttatagcagccgtcgaacggttgtgggattatggcatgagctcaagtccacatgtactgagaaatagggcgcagtcaggaagcgtaccccagttt
aagaaggttgtattccaagaattcacagacggtagctttacccagccgctttatcgaggagagttgaatgagcaccttggtttgctgggaccgtac
atccgcgcagaagtcgaagacaatataatggtcacctttcggaaccaagcctccaggccatacagtttctacagttctctgatctcatacgaggaa
gatcagaggcaaggagcagaaccaaggaagaacttcgtgaaaccaaacgagacaaagacctatttctggaaagttcagcatcatatggcacccact
aaagatgagtttgactgcaaagcctgggcttatttctctgatgttgacctggaaaaagatgtgcactcaggcctgattggaccccttctggtctgc
cacactaacacactgaaccctgctcatgggagacaagtgacagtacaggaatttgctctgtttttcaccatctttgatgagaccaaaagctggtac
ttcactgaaaatatggaaagaaactgcagggctccctgcaatatccagatggaagatcccacttttaaagagaattatcgcttccatgcaatcaat
ggctacataatggatacactacctggcttagtaatggctcaggatcaaaggattcgatggtatctgctcagcatgggcagcaatgaaaacatccat
tctattcatttcagtggacatgtgttcactgtacgaaaaaaagaggagtataaaatggcactgtacaatctctatccaggtgtttttgagacagtg
gaaatgttaccatccaaagctggaatttggcgggtggaatgccttattggcgagcatctacatgctgggatgagcacactttttctggtgtacagc
aataagtgtcagactcccctgggaatggcttctggacacattagagattttcagattacagcttcaggacaatatggacagtgggccccaaagctg
gccagacttcattattccggatcaatcaatgcctggagcaccaaggagcccttttcttggatcaaggtggatctgttggcaccaatgattattcac
ggcatcaagacccagggtgcccgtcagaagttctccagcctctacatctctcagtttatcatcatgtatagtcttgatgggaagaagtggcagact
tatcgaggaaattccactggaaccttaatggtcttctttggcaatgtggattcatctgggataaaacacaatatttttaaccctccaattattgct
cgatacatccgtttgcacccaactcattatagcattcgcagcactcttcgcatggagttgatgggctgtgatttaaatagttgcagcatgccattg
ggaatggagagtaaagcaatatcagatgcacagattactgcttcatcctactttaccaatatgtttgccacctggtctccttcaaaagctcgactt
cacctccaagggaggagtaatgcctggagacctcaggtgaataatccaaaagagtggctgcaagtggacttccagaagacaatgaaagtcacagga
gtaactactcagggagtaaaatctctgcttaccagcatgtatgtgaaggagttcctcatctccagcagtcaagatggccatcagtggactctctt
ttttcagaatggcaaagtaaaggtttttcagggaaatcaagactccttcacacctgtggtgaactctctagacccaccgttactgactcgctacct
tcgaattcacccccagagttgggtgcaccagattgccctgaggatggaggttctgggctgcgaggcacaggacctctactga.
SEQ ID NO: 6: (a nucleic acid sequence encoding a full-length F8-BDD-299 protein)
atgcagatcgaactgagcacctgcttcttcctgtgtctcctgagattctgctttagtgctaccagacggtattacctgggagccgtcgagctg
agttgggattacatgcagtccgacctcggagaactgcctgtggatgcacgctttccaccaagagtgcctaagtcattcccattcaacacctcagtc
gtgtataagaagactctgttcgtcgagtttactgatcacctgttcaatatcgctaaacctagaccaccctggatgggactgctgggtcctacaatc
caggcagaggtctatgacactgtggtgattacacttaagaacatggcttcccatcctgtcagtcttcatgctgttggtgtatcctactggaaagct
tctgagggagctgaatatgatgatcagaccagtcaaagggagaaagaagatgataaagtcttccctggtggaagccatacatatgtctggcaggtc
ctgaaagagaatggtccaatggcctctgacccactgtgccttacctactcatatctttctcatgtggacctggtaaaagacttgaattcaggcctc
attggagccctactagtatgtagagaagggagtctggccaaggaaaagacacagaccttgcacaaatttatactactttttgctgtatttgatgaa
gggaaaagttggcactcagaaacaaagaactccttgatgcaggatagggatgctgcatctgctcgggcctggcctaaaatgcacacagtcaatgg
ttatgtaaacaggtctctgccaggtctgattggatgccacaggaaatcagtctattggcatgtgattggaatgggcaccactcctgaagtgcactc
aatattcctcgaaggtcacacatttcttgtgaggaaccatcgccaggcgtccttggaaatctcgccaataactttccttactgctcaaacactctt
gatggaccttggacagtttctactgttttgtcatatctcttcccaccaacatgatggcatggaagcttatgtcaaagtagacagctgtccagagga
accccaactacgaatgaaaaataatgaagaagcggaagactatgatgatgatcttactgattctgaaatggatgtggtcaggtttgatgatgacaa
ctctccttcctttatccaaattcgctcagttgccaagaagcatcctaaaacttgggtacattacattgctgctgaagaggaggactgggactatgc
tcccttagtcctcgcccccgatgacagaagttataaaagtcaatatttgaacaatggccctcagcggattggtaggaagtacaaaaaagtccgatt
tatggcatacacagatgaaacctttaagactcgtgaagctattcagcatgaatcaggaatcttgggacctttactttatggggaagttggagacac
actgttgattatatttaagaatcaagcaagcagaccatataacatctaccctcacggaatcactgatgtccgtcctttgtattcaaggagattacc
aaaaggtgtaaaacatttgaaggattttccaattctgccaggagaaatattcaaatataaatggacagtgactgtagaagatgggccaactaaatc
agatcctcggtgcctgacccgctattactctagtttcgttaatatggagagagatctagcttcaggactcattggccctctcctcatctgctacaa
agaatctgtagatcaaagaggaaaccagataatgtcagacaagaggaatgtcatcctgttttctgtatttgatgagaaccgaagctggtacctcac
agagaatatacaacgctttctccccaatccagctggagtgcagcttgaggatccggagtttcaggcatccaatatcatgcattctataaacgggta
tgtatttgattctttgcagttgagtgtgtgtctgcatgaggttgcctactggtacattctgtctataggggcgcagacggatttcctttcagtgtt
cttcagcgggtatacatttaaacataagatggtatatgaggacaccttgacattgtttccattttccggcgaaaccgtattcatgtcaatggagaa
cccagggttgtggatactcggttgccataatagtgacttcagaaaccgagggatgacggcccttctcaaagtaagttcatgtgataagaataccgg
tgattactacgaagatagctatgaggatattagcgcctacttgcttagcaagaataacgctattgaacctaggagcttttcccagaatagccgaca
tcctagcactcgccaaaaacagtttaatgcgacaactatccctgagaacgatattgagaaaactgatccctggtttgcacatcgcactcctatgcc
aaagatccaaaacgtgagcagctctgacctccttatgttgcttagacaatctcccacacctcatggactctcactttccgatctgcaggaggcgaa
gtatgaaaccttctcagacgacccatccccaggagccatagactcaaacaatagtctctcagaaatgacgcactttagacctcaactccatcacag
tggggatatggtatttacccccgagagtggtctgcagcttaggcttaatgaaaaattgggaaccaccgctgcaacggaactcaaaaaactggact
tcaaggtttctagcacgtcaaacaatcttatatcaaccataccatccgacaaccttgccgcaggaaccgataacacatcaagcctggggcctccat
caatgccggtgcactatgattcacagttggatactaccctcttcgggaagaaaagttcaccgctgactgaaagcggtggcccactgtctctgagtg
aagagaataatgattctaaacttctcgagagcggcctcatgaatagtcaggagagttcttgggggaaaaatgttagcagtactgagagcggacgg
ctcttcaaaggtaagcgggcacatgggcccgctcttctgactaaggataacgctttgttcaaagttagcatatcactcctgaaaactaacaagac
ctcaaataattctgcaacgaaccggaagacccatattgacggtccaagtttgctcatcgagaactccccgagtgtatggcagaacattcttgagag
cgataccgagtttcccccggtactcaagaggcatcagcgggagattacgcgaaccacactccagtccgatcaggaagaaattgattatgacgata
ctatcagtgtagagatgaaaaaagaagactttgacatctatgatgaggacgagaaccagtctccacgaagctttcagaaaaaaacaaggcactat
tttatcgccgctgttgaacggctgtgggactacggtatgtcctcttcaccccacgtgctgcggaaccgggcccagtcaggctcagtaccccaattc
aagaaggtggtattccaggaatttaccgatggatctttcacgcaacctctttaccgaggtgagctgaacgaacatcttggccttctcggtccttat
attagagcagaggtggaagacaatataatggtcacctttcggaaccaagcctccaggccatacagtttctacagttctctgatctcatacgaggaa
gatcagaggcaaggagcagaaccaaggaagaacttcgtgaaaccaaacgagacaaagacctatttctggaaagttcagcatcatatggcacccact
aaagatgagtttgactgcaaagcctgggcttatttctctgatgttgacctggaaaaagatgtgcactcaggcctgattggaccccttctggtctgc
cacactaacacactgaaccctgctcatgggagacaagtgacagtacaggaatttgctctgtttttcaccatctttgatgagaccaaaagctggtac
ttcactgaaaatatggaaagaaactgcagggctccctgcaatatccagatggaagatcccacttttaaagagaattatcgcttccatgcaatcaat
ggctacataatggatacactacctggcttagtaatggctcaggatcaaaggattcgatggtatctgctcagcatgggcagcaatgaaaacatcca
ttctattcatttcagtggacatgtgttcactgtacgaaaaaaagaggagtataaaatggcactgtacaatctctatccaggtgtttttgagacag
tggaaatgttaccatccaaagctggaatttggcgggtggaatgccttattggcgagcatctacatgctgggatgagcacactttttctggtgtaca
gcaataagtgtcagactcccctgggaatggcttctggacacattagagattttcagattacagcttcaggacaatatggacagtgggccccaaag
ctggccagacttcattattccggatcaatcaatgcctggagcaccaaggagcccttttcttggatcaaggtggatctgttggcaccaatgattat
tcacggcatcaagacccagggtgcccgtcagaagttctccagcctctacatctctcagtttatcatcatgtatagtcttgatgggaagaagtggca
gacttatcgaggaaattccactggaaccttaatggtcttctttggcaatgtggattcatctgggataaaacacaatatttttaaccctccaatta
ttgctcgatacatccgtttgcacccaactcattatagcattcgcagcactcttcgcatggagttgatgggctgtgatttaaatagttgcagcatg
ccattgggaatggagagtaaagcaatatcagatgcacagattactgcttcatcctactttaccaatatgtttgccacctggtctccttcaaaagc
tcgacttcacctccaagggaggagtaatgcctggagacctcaggtgaataatccaaaagagtggctgcaagtggacttccagaagacaatgaaag
tcacaggagtaactactcagggagtaaaatctctgcttaccagcatgtatgtgaaggagttcctcatctccagcagtcaagatggccatcagtgg
actctcttttttcagaatggcaaagtaaaggtttttcagggaaatcaagactccttcacacctgtggtgaactctctagacccaccgttactgact
cgctaccttcgaattcacccccagagttgggtgcaccagattgccctgaggatggaggttctgggctgcgaggcacaggacctctactga.

The gene encoding the recombinant coagulation factor VIII of the present application is rich in sequences of glycosylation sites, and the expressed coagulation factor VIII (F8 protein) has a good coagulation function, is easily secreted outside a cell and easily interacts with a cofactor such as thrombin (FIIa). In addition, the expressed coagulation factor VIII has a weak ability to induce an antibody response and can effectively ensure a treatment effect, reduce a risk of immune rejection and lower a treatment cost.

In a third aspect, the present application provides a recombinant expression vector. The recombinant expression vector includes the gene encoding the recombinant coagulation factor VIII according to the second aspect.

Preferably, the recombinant expression vector includes a viral vector or a plasmid vector containing the gene encoding the recombinant coagulation factor VIII according to the second aspect.

Preferably, the viral vector includes a lentiviral vector pEGWI.

Preferably, a 5′ splice donor site GT of the lentiviral vector pEGWI is mutated into CA.

Preferably, an enhancer in a U3 region of the lentiviral vector pEGWI is deleted.

Preferably, the U3 region of the lentiviral vector pEGWI contains an insulator.

Preferably, the recombinant expression vector includes an EF1α promoter.

In the present application, the lentiviral vector pEGWI is modified: a wild-type 5′ splice donor site GT is mutated into CA, the enhancer in the U3 region is deleted, and an insulator (cHS4) is added to the U3 region, which can effectively improve the transduction efficiency and expression efficiency of pEGWI to reduce a vector production cost and can also improve safety.

In a fourth aspect, the present application provides a recombinant lentivirus. The recombinant lentivirus includes the gene encoding the recombinant coagulation factor VIII according to the second aspect.

Preferably, a method for preparing the recombinant lentivirus includes:
packaging the lentiviral vector according to the third aspect using a packaging plasmid(s) to obtain the recombinant lentivirus.

Preferably, the packaging plasmid(s) include(s) pNHP and pHEF-VSV-G.

Preferably, the method for preparing the recombinant lentivirus includes the following steps:
(1) co-transfecting the lentiviral vector according to the third aspect and the packaging plasmids pNHP and pHEF-VSV-G into a mammalian cell HEK293T and culturing the mammalian cell HEK293T for 24-72 h; and
(2) performing purification and concentration to obtain the recombinant lentivirus.

In a fifth aspect, the present application provides a recombinant cell. The recombinant cell includes the gene encoding the recombinant coagulation factor VIII according to the second aspect.

Preferably, a genome of the recombinant cell is integrated with the gene encoding the recombinant coagulation factor VIII according to the second aspect.

Preferably, the recombinant cell contains the recombinant expression vector according to the third aspect.

In a sixth aspect, the present application provides a method for preparing the recombinant cell according to the fifth aspect. The method includes:

introducing the gene encoding the recombinant coagulation factor VIII according to the second aspect, the recombinant expression vector according to the third aspect or the recombinant lentivirus according to the fourth aspect into a host cell to obtain the recombinant cell.

Preferably, the introduction is carried out by a method which includes any one of electrical transduction, a viral vector system, a non-viral vector system or direct gene injection.

Preferably, the host cell includes a hematopoietic stem cell.

In a seventh aspect, the present application provides a pharmaceutical composition. The pharmaceutical composition includes any one or a combination of the recombinant coagulation factor VIII according to the first aspect, the gene encoding the recombinant coagulation factor VIII according to the second aspect, the recombinant expression vector according to the third aspect, the recombinant lentivirus according to the fourth aspect or the recombinant cell according to the fifth aspect.

Preferably, the pharmaceutical composition further includes any one or a combination of at least two of a pharmaceutically acceptable carrier, excipient or diluent.

In an eighth aspect, the present application provides a use of the recombinant coagulation factor VIII according to the first aspect, the gene encoding the recombinant coagulation factor VIII according to the second aspect, the recombinant expression vector according to the third aspect, the recombinant lentivirus according to the fourth aspect, the recombinant cell according to the fifth aspect or the pharmaceutical composition according to the seventh aspect to preparation of a medicament for treating hemophilia.

Compared with the existing art, the present application has the beneficial effects below.

(1) Through gene modification, the recombinant coagulation factor VIII of the present application is rich in glycosylation sites, has a good coagulation function, is easily secreted outside a cell and easily interacts with a cofactor such as thrombin (FIIa). In addition, the recombinant coagulation factor VIII has a weak response to an antibody and can effectively ensure a treatment effect, reduce a risk of immune rejection and lower a treatment cost.

(2) In the present application, the lentiviral vector pEGWI is modified, which can effectively improve the transduction efficiency and expression efficiency of pEGWI to reduce a vector cost and can also improve safety.

(3) In the present application, an expression vector constructed by using the gene encoding the recombinant coagulation factor VIII and a lentiviral vector can perform successful expression in vivo in HA mice and can correct a bleeding phenotype of HA mice to a certain extent. The expression vector has a weak response to an antibody and is of great significance for ensuring the effectiveness of gene therapy, which lays a basis for relieving HA symptoms faster and achieving a more comprehensive and long lasting gene therapy effect.

### BRIEF DESCRIPTION OF DRAWINGS

FIG. 1 is a structural diagram of a lentiviral vector pEGWI;

FIG. 2A is a structural diagram of a normal F8-BDD gene;

FIG. 2B is a structural diagram of an F8-BDD-N8 gene;

FIG. 2C is a structural diagram of an F8-BDD-299 gene;

FIG. 2D is a structural diagram of a recombinant lentiviral vector;

FIG. 3 shows a viral copy number of a recombinant lentivirus;

FIG. 4 is a flowchart of an analysis of the protein expression of cells transfected with a recombinant lentivirus;

FIG. 5 is a graph showing the results of the protein expression of cells transduced with a recombinant lentivirus;

FIG. 6 is a graph showing the detection results of in vitro plasma by an APTT assay;

FIG. 7 is a graph showing the detection results of in vitro plasma by a substrate luminescence assay;

FIG. 8 is a graph showing the Western blotting results of factor VIII treated with a glycosylation inhibitor;

FIG. 9 is a graph showing the in vitro activity of factor VIII;

FIG. 10 is a flowchart of the treatment of HA mice;

FIG. 11 is a graph showing the in vivo activity of factor VIII in mice;

FIG. 12 is a graph showing the detection results of mouse plasma by an APTT assay; and

FIG. 13 is a graph showing the detection results of mouse plasma by an enzyme-linked immunosorbent assay.

### DETAILED DESCRIPTION

To further elaborate on the technical means adopted and effects achieved in the present application, the present application is further described below in conjunction with examples and drawings. It is to be understood that the specific embodiments described herein are intended to illustrate the present application and not to limit the present application.

Experiments without specific techniques or conditions noted in the examples are conducted according to techniques or conditions described in the literature in the art or a product specification. The reagents or instruments used herein without manufacturers specified are conventional products commercially available from proper channels.

### Example 1

A lentiviral vector was constructed. The method specifically includes the steps below.

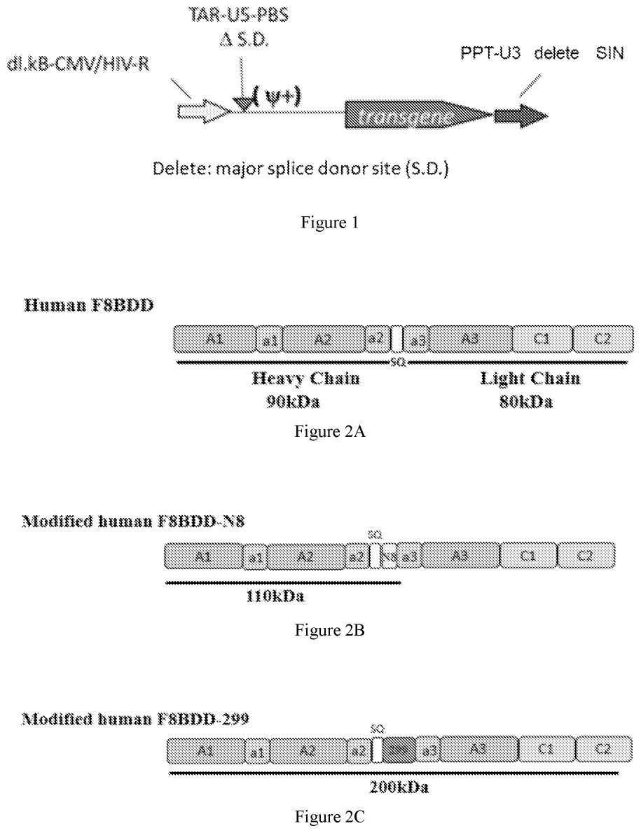

(1) The structural diagram of a lentiviral vector pEGWI is shown in FIG. 1. The wild-type 5′ splice donor site GT was mutated into CA, the enhancer in U3 was deleted, and an insulator (cHS4) was added to U3. For a specific modification method, refer to “Contributions of Viral Splice Sites and cis-Regulatory Elements to Lentivirus Vector Function, Cui et al. Journal of Virology, July 1999, p. 6171-6176”.

(2) A promoter and an F8-BDD/F8-BDD-N8/F8-BDD-299 gene were inserted.

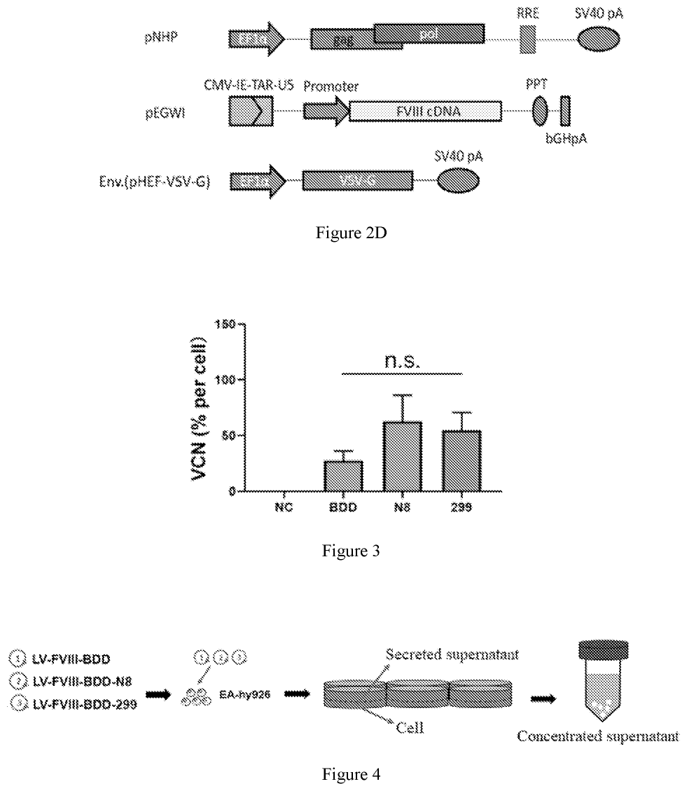

A normal unmodified F8-BDD gene sequence (as shown in SEQ ID NO: 7), an F8-BDD-N8 gene sequence (as shown in SEQ ID NO: 5) and an F8-BDD-299 gene sequence (as shown in SEQ ID NO: 6) were chemically synthesized with the selected possible sequences of glycosylation sites, and a human EF1α (hEF1α) promoter sequence was added. The gene structure of normal F8-BDD is shown in FIG. 2A. F8-BDD-N8 and F8-BDD-299 were inserted with synthesized glycosylation site-related sequences separately and their gene structures are shown in FIGS. 2B and 2C. Normal F8-BDD, F8-BDD-N8 and F8-BDD-299 were inserted into lentiviral vectors pEGWI through restriction enzyme digestion sites. The obtained products were identified by sequencing and double digestion (for best reaction conditions, refer to the original recommendations of New England Biolab [NEB]), where the BamHI cloning site (ggatcc-acc)-AUG was used for the 5′ end and the SpeI cloning site (actagt) was used for the 3′ end, to obtain correctly linked lentiviral vectors carrying the normal F8-BDD, F8-BDD-N8 or F8-BDD-299 gene regulated by hEF1α. Specific linkage positions and the composition of the lentiviral vectors are shown in FIG. 2D.

SEQ ID NO: 7:
atgcagatcgaactgagcacctgcttcttcctgtgtctcctgagattctgctttagtgctaccagacggtattacctgggagccgtcgagctg
agttgggattacatgcagtccgacctcggagaactgcctgtggatgcacgctttccaccaagagtgcctaagtcattcccattcaacacctcagtc
gtgtataagaagactctgttcgtcgagtttactgatcacctgttcaatatcgctaaacctagaccaccctggatgggactgctgggtcctacaatc
caggcagaggtctatgacactgtggtgattacacttaagaacatggcttcccatcctgtcagtcttcatgctgttggtgtatcctactggaaagct
tctgagggagctgaatatgatgatcagaccagtcaaagggagaaagaagatgataaagtcttccctggtggaagccatacatatgtctggcaggtc
ctgaaagagaatggtccaatggcctctgacccactgtgccttacctactcatatctttctcatgtggacctggtaaaagacttgaattcaggcct
cattggagccctactagtatgtagagaagggagtctggccaaggaaaagacacagaccttgcacaaatttatactactttttgctgtatttgatg
aagggaaaagttggcactcagaaacaaagaactccttgatgcaggatagggatgctgcatctgctcgggcctggcctaaaatgcacacagtcaat
ggttatgtaaacaggtctctgccaggtctgattggatgccacaggaaatcagtctattggcatgtgattggaatgggcaccactcctgaagtgca
ctcaatattcctcgaaggtcacacatttcttgtgaggaaccatcgccaggcgtccttggaaatctcgccaataactttccttactgctcaaacact
cttgatggaccttggacagtttctactgttttgtcatatctcttcccaccaacatgatggcatggaagcttatgtcaaagtagacagctgtccaga
ggaaccccaactacgaatgaaaaataatgaagaagcggaagactatgatgatgatcttactgattctgaaatggatgtggtcaggtttgatgatg
acaactctccttcctttatccaaattcgctcagttgccaagaagcatcctaaaacttgggtacattacattgctgctgaagaggaggactgggact
atgctcccttagtcctcgcccccgatgacagaagttataaaagtcaatatttgaacaatggccctcagcggattggtaggaagtacaaaaaagtc
cgatttatggcatacacagatgaaacctttaagactcgtgaagctattcagcatgaatcaggaatcttgggacctttactttatggggaagttgg
agacacactgttgattatatttaagaatcaagcaagcagaccatataacatctaccctcacggaatcactgatgtccgtcctttgtattcaaggag
attaccaaaaggtgtaaaacatttgaaggattttccaattctgccaggagaaatattcaaatataaatggacagtgactgtagaagatgggccaa
ctaaatcagatcctcggtgcctgacccgctattactctagtttcgttaatatggagagagatctagcttcaggactcattggccctctcctcatc
tgctacaaagaatctgtagatcaaagaggaaaccagataatgtcagacaagaggaatgtcatcctgttttctgtatttgatgagaaccgaagctg
gtacctcacagagaatatacaacgctttctccccaatccagctggagtgcagcttgaggatccagagttccaagcctccaacatcatgcacagca
tcaatggctatgtttttgatagtttgcagttgtcagtttgtttgcatgaggtggcatactggtacattctaagcattggagcacagactgacttc
ctttctgtcttcttctctggatataccttcaaacacaaaatggtctatgaagacacactcaccctattcccattctcaggagaaactgtcttcat
gtcgatggaaaacccaggtctatggattctggggtgccacaactcagactttcggaacagaggcatgaccgccttactgaaggtttctagttgtg
acaagaacactggtgattattacgaggacagttatgaagatatttcagcatacttgctgagtaaaaacaatgccattgaaccaagaagcttttct
cagaatcctcctgtcctcaaacgccatcaacgggagattacacggaccacactccaaagcgatcaggaggagatcgactatgacgataccatatc
tgtggaaatgaagaaagaggacttcgacatctacgacgaagatgagaaccaaagtccaagatccttccagaagaagactaggcactacttcatcg
ctgccgtggaacgcctctgggattacggaatgtccagttctccacatgtcctcaggaatagggcacagtctggctctgttccacagtttaagaaa
gttgtctttcaggagttcacagatggctcattcactcaaccactgtatagaggcgaactgaatgagcacctgggactgctgggtccctacatca
gagccgaagtggaggataacattatggtcacctttcggaaccaagcctccaggccatacagtttctacagttctctgatctcatacgaggaagat
cagaggcaaggagcagaaccaaggaagaacttcgtgaaaccaaacgagacaaagacctatttctggaaagttcagcatcatatggcacccactaa
agatgagtttgactgcaaagcctgggcttatttctctgatgttgacctggaaaaagatgtgcactcaggcctgattggaccccttctggtctgcc
acactaacacactgaaccctgctcatgggagacaagtgacagtacaggaatttgctctgtttttcaccatctttgatgagaccaaaagctggtac
ttcactgaaaatatggaaagaaactgcagggctccctgcaatatccagatggaagatcccacttttaaagagaattatcgcttccatgcaatcaa
tggctacataatggatacactacctggcttagtaatggctcaggatcaaaggattcgatggtatctgctcagcatgggcagcaatgaaaacatc
cattctattcatttcagtggacatgtgttcactgtacgaaaaaaagaggagtataaaatggcactgtacaatctctatccaggtgtttttgagac
agtggaaatgttaccatccaaagctggaatttggcgggtggaatgccttattggcgagcatctacatgctgggatgagcacactttttctggtgt
acagcaataagtgtcagactcccctgggaatggcttctggacacattagagattttcagattacagcttcaggacaatatggacagtgggcccca
aagctggccagacttcattattccggatcaatcaatgcctggagcaccaaggagcccttttcttggatcaaggtggatctgttggcaccaatgat
tattcacggcatcaagacccagggtgcccgtcagaagttctccagcctctacatctctcagtttatcatcatgtatagtcttgatgggaagaagt
ggcagacttatcgaggaaattccactggaaccttaatggtcttctttggcaatgtggattcatctgggataaaacacaatatttttaaccctcca
attattgctcgatacatccgtttgcacccaactcattatagcattcgcagcactcttcgcatggagttgatgggctgtgatttaaatagttgcag
catgccattgggaatggagagtaaagcaatatcagatgcacagattactgcttcatcctactttaccaatatgtttgccacctggtctccttcaa
aagctcgacttcacctccaagggaggagtaatgcctggagacctcaggtgaataatccaaaagagtggctgcaagtggacttccagaagacaatg
aaagtcacaggagtaactactcagggagtaaaatctctgcttaccagcatgtatgtgaaggagttcctcatctccagcagtcaagatggccatca
gtggactctcttttttcagaatggcaaagtaaaggtttttcagggaaatcaagactccttcacacctgtggtgaactctctagacccaccgttac
tgactcgctaccttcgaattcacccccagagttgggtgcaccagattgccctgaggatggaggttctgggctgcgaggcacaggacctctactga.

### Example 2

In this example, the lentiviral vectors constructed in Example 1 were further packaged, purified and concentrated to obtain recombinant lentiviruses. For the experimental method, refer to [1] Chang L-J, Urlacher V, Iwakuma T, et al. Efficacy and safety analyses of a recombinant human immunodeficiency virus type 1 derived vector system[J]. Gene Therapy, 1999, 6(5): 715-728. [2] Chang L J, Zaiss A K. Chang, L-J and Zaiss, AK. Lentiviral vectors. Preparation and use. Methods Mol Med 69: 303-318 [J]. Methods in Molecular Medicine, 2002, 69: 303-318.

For specific steps, refer to the literature listed above. The experimental method is briefly described as follows.

(1) The lentiviral vectors constructed in Example 1 and packaging plasmids pNHP and pHEF-VSV-G were co-transfected into mammalian cells HEK293T, the mammalian cells HEK293T were then cultured for 48 h, and the viral vector supernatant was collected.

(2) The lentiviruses collected from the culture were purified and concentrated to obtain the recombinant lentiviruses.

(3) The viral copy number (VCN) of each lentivirus was detected. The detection results are shown in FIG. 3. With the same multiplicity of infection, LV-F8-BDD, LV-F8-BDD-N8 and LV-F8-BDD-299 lentiviruses have basically similar copy number.

### Example 3

In this example, the recombinant lentiviruses prepared in Example 2 were tested in vitro.

Three types of lentivirus (LV-F8-BDD, LV-F8-BDD-N8 and LV-F8-BDD-299) carrying the normal F8-BDD, F8-BDD-N8 or F8-BDD-299 gene and prepared in Example 2 were transduced into EA-hy 926 endothelial cells, separately. The lentivirus was transduced by the method below.

A DMEM medium containing 10% fetal bovine serum and 1% penicillin-streptomycin solution was added to a six-well plate (Corning, USA). 4×10+EA-hy 926 endothelial cell lines were inoculated per well, incubated for 18 h at 37° ° C. under 5% CO2 and transfected with the lentivirus at an MOI of 50. Polybrene (8 μg/mL, Sigma-Aldrich) was supplemented until the final volume of the medium was 600 μL. The transduction was performed for 24 h, and then the medium was replaced with a fresh medium every day until the cell confluence reached 90.0%. The cells were transferred to a T75 flask (Corning, USA).

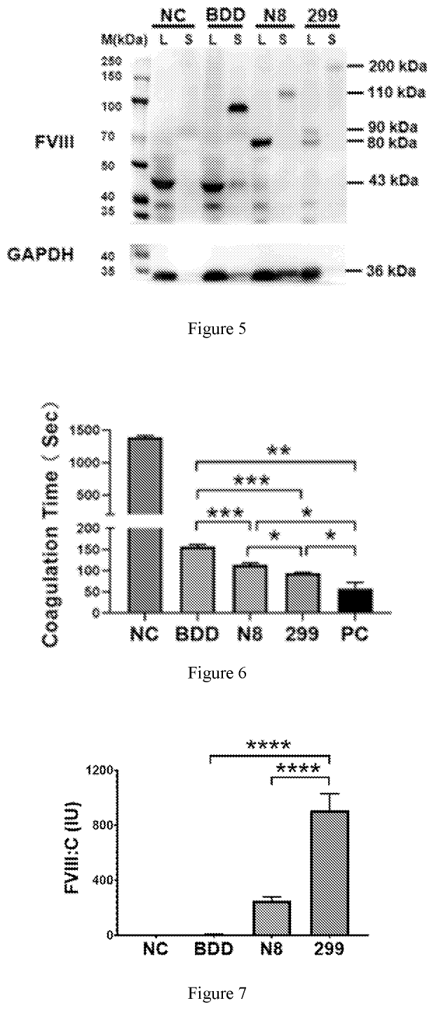

Protein expression was detected to determine the expression of F8-BDD, F8-BDD-N8 and F8-BDD-299 genes in cells. The specific process is shown in FIG. 4. The supernatant secreted by the transduced EA-hy 926 cells was collected and concentrated, and the intracellular extract was also collected. The protein expression was detected through Western blotting. The cells transduced with no lentivirus were used as a negative control (NC). With GAPDH as internal reference, the results are shown in FIG. 5, where L denotes the detection result of the cell extract and S denotes the detection result of the cell culture supernatant. As shown in FIG. 5, a small amount of factor VIII (F8 protein) was expressed in the cells transduced with no lentivirus (low activity, 43 kDa), while a relatively large amount of F8-BDD, F8-BDD-N8 or F8-BDD-299 was obviously expressed in the cells transduced with the lentivirus carrying the normal F8-BDD, F8-BDD-N8 or F8-BDD-299 gene; and relative to F8-BDD, more complete F8 proteins (110-200 kDa) can be obtained in the supernatant of the cells transduced with F8-BDD-N8 or F8-BDD-299. It indicates that factor VIII expressed by the genetically modified F8-BDD genes: F8-BDD-N8 and F8-BDD-299 in the present application is more likely to be secreted outside a cell, ensuring effective coagulation.

The coagulation function is mainly evaluated by two methods: an activated partial thromboplastin time (APTT) assay and a substrate luminescence assay. The APTT (Siemens Healthcare Diagnostics Products GmbH, Germany) assay was conducted as follows: at 37° C., 50 μL of the cell supernatant was added to 50 μL of the plasma to be tested, then 100 μL of Actin reagent (factor XII activator and cerebral phospholipid) in the APTT was added, thoroughly mixed, and incubated for 3 min at 7° C., and finally 100 μL of calcium ions (CaCl₂)) was added to observe the time required for plasma coagulation, that is, activated partial thromboplastin time.

The substrate luminescence assay is a method for determining activity with an F8 chromogenic assay kit (Hyphen BioMed, France). The substrate luminescence assay was conducted as follows: the plasma to be tested and the blank control group were diluted 40 times with a Tris-BSA buffer (R4+), 50 μL was added to a microplate, added with 50 μL of factor X (R1), 50 μL of an activated factor IX mixture (R2) and 50 μL of SXa-11 substrate (R3) separately and incubated for 5 min at 37° C., and 50 μL of 20% acetic acid was added to stop the reaction. The absorbance was read at 405 nm.

The above-collected supernatant of the virus-transduced EA-hy 926 cells was taken out from −80° C. and thawed on ice. Each supernatant was mixed with the plasma of an F8 deficient patient. The plasma of the F8 deficient patient was used as a negative control (NC) and the plasma of a healthy volunteer was used as a positive control (PC). The detection was performed by the APTT assay and the substrate luminescence assay.

The detection results of the APTT assay are shown in FIG. 6. Compared with the negative control, the supernatant of the cells transduced with F8-BDD, the supernatant of the cells transduced with F8-BDD-N8 and the supernatant of the cells transduced with F8-BDD-299 each have good coagulation activity and a significant coagulation effect in that the plasma coagulation time is significantly reduced. The supernatant of the cells transduced with F8-BDD-299 has the best effect in that the coagulation time is 94.3 s, which has a certain statistical difference (p<0.05) compared with that (57.5 s) of the positive control. The coagulation time of the supernatant of the cells transduced with F8-BDD-N8 is 113.7 s, which has a certain statistical difference (p average)<0.05) compared with those of F8-BDD-299 and the positive control. The coagulation time of the supernatant of the cells transfected with F8-BDD is 156.7 s, which has a relatively large statistical difference (p average)<0.001) compared with those of F8-BDD-N8, F8-BDD-299 and the positive control. It indicates that factor VIII expressed by the modified F8-BDD genes: F8-BDD-N8 and F8-BDD-299 in the present application has a good coagulation effect.

FIG. 7 shows the detection results of the substrate luminescence assay. The activity was obtained using the F8 chromogenic assay kit and then factor VIII was quantified through an ELISA to obtain unit activity. The unit activity of factor VIII expressed by F8-BDD-299 is 100 times that of factor VIII expressed by F8-BDD and 2.8 times that of factor VIII expressed by F8-BDD-N8.

To sum up, in the present application, the lentiviral vectors are constructed using the modified F8-BDD genes: F8-BDD-N8 and F8-BDD-299, respectively and successfully expressed in cells, and factor VIII expressed by F8-BDD-N8 and F8-BDD-299 is more easily secreted outside a cell and has a good coagulation effect.

### Example 4

In this example, the N-glycosylation of factor VIII was detected.

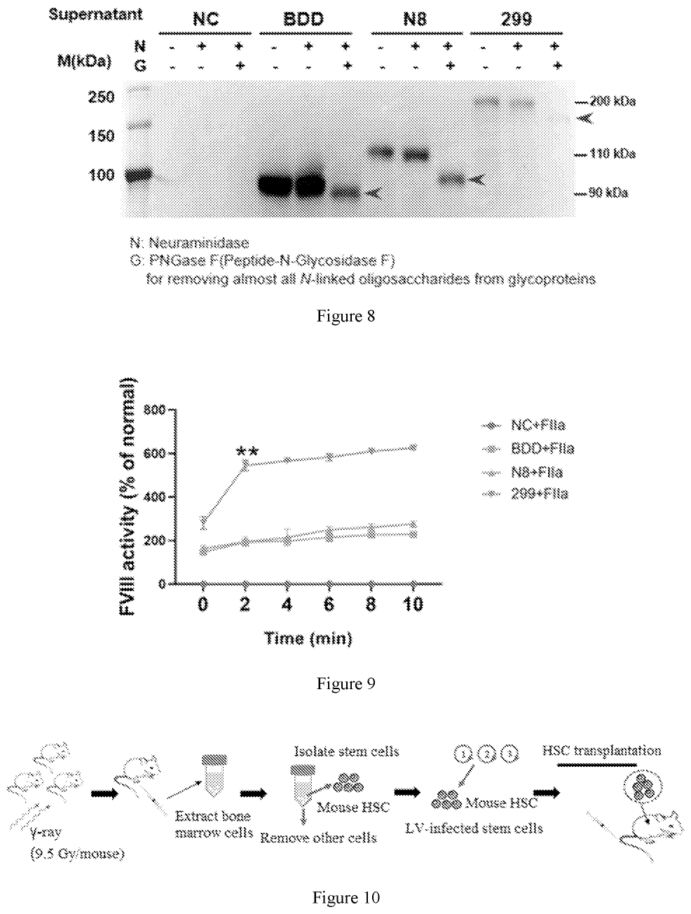

The supernatants of endothelial cells transduced with F8-BDD, F8-BDD-N8 and F8-BDD-299 were separately treated with glycosylation inhibitors: neuraminidase (N) and peptide-N-glycosidase F (G). The glycosylation inhibitors can induce deglycosylation and reduce a molecular weight. The results of Western blotting are shown in FIG. 8, where “+” denotes treatment with the corresponding enzyme and “−” denotes no treatment with the corresponding enzyme. After treatment with the glycosylation inhibitors, the protein band with a reduced protein molecular weight after deglycosylation is shown by an arrow. The molecular weight of factor VIII expressed by F8-BDD-N8 and F8-BDD-299 is significantly reduced (++ lanes compared with − lanes), while the molecular weight of factor VIII expressed by F8-BDD is not reduced, indicating that the modification of the F8-BDD gene in the present application can effectively introduce glycosylation sites.

Thrombin (FIIa) is an important cofactor for factor VIII. FIIa is used for treating the supernatant of the transduced endothelial cells to investigate whether the modification of the B domain also affects the interaction with a procoagulant cofactor. The addition of thrombin enhances the activation of F8, and more proteins (with a molecular weight of 43 kDa) associated with the A2 region of factor VIII are produced. After thrombin was added, activity detection was performed every two minutes (by the substrate luminescence assay as described above). The results are shown in FIG. 9. From the second minute, the activity of factor VIII expressed by F8-BDD-299 is significantly increased and the activity of factor VIII expressed by F8-BDD and F8-BDD-N8 is slightly increased.

### Example 5

Three types of lentivirus (LV-F8-BDD, LV-F8-BDD-N8 and LV-F8-BDD-299) carrying F8-BDD, F8-BDD-N8 or F8-BDD-299 were prepared as shown in Example 2 and were transduced into hematopoietic stem cells of mice. HA mice were treated as shown in FIG. 10. C57BL/6 mice (which were six weeks old and purchased from Beijing Biosubstrate Technologies) with the knockout of the F8 gene were used as HA mice. All the mice were placed in a pathogen-free environment and irradiated (9.5 Gy/mouse) with an x-ray irradiation cabinet (Faxitron, Tucson, AZ, USA). Bone marrow cells were collected from the tibia and femur of the HA mice. The hematopoietic stem cells were isolated from the bone marrow cells and transduced with the lentiviruses LV-F8-BDD, LV-F8-BDD-N8 and LV-F8-BDD-299 respectively to obtain stem cells carrying the F8-BDD, F8-BDD-N8 or F8-BDD-299 gene. The transduced stem cells were injected back to HA mice through intravenous injection for disease treatment.

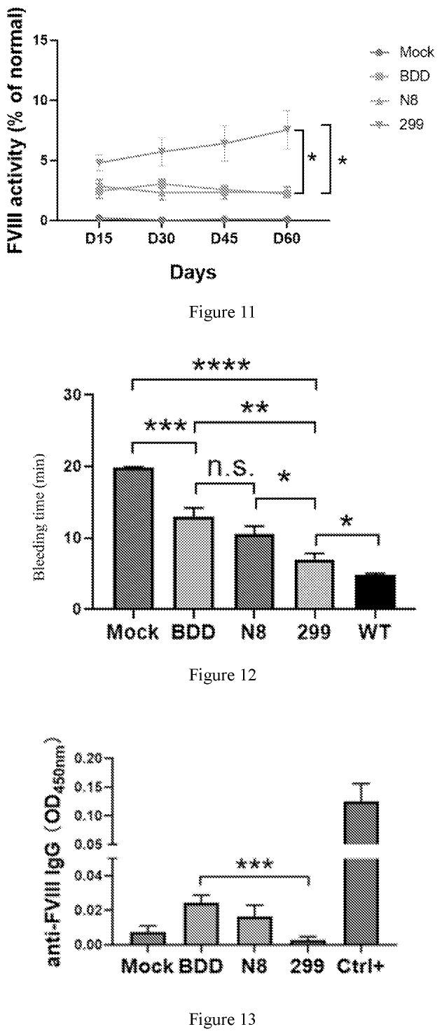

On Day 15, Day 30, Day 45 and Day 60 after allogeneic bone marrow transplantation, the blood was taken from mice and the plasma was isolated from the blood. The activity of factor VIII in the plasma was determined by a two-step substrate luminescence assay with untreated hemophilia mice (Mock) and wild-type mice (WT) as controls. The results are shown in FIG. 11. The activity of factor VIII expressed by F8-BDD-299 is continuously increased from 5% on Day 15 to 8% on Day 60. The activity of factor VIII expressed by F8-BDD and F8-BDD-N8 remains to be about 3.0%, which has a certain statistical difference (P<0.05) with that of F8-BDD-299. The coagulation function of the plasma was determined by the APTT assay. The results are shown in FIG. 12. The coagulation time of factor VIII expressed by F8-BDD-299 is shortest and closest to that of wild-type mice (p<0.05).

In addition, for a response of an antibody, the orbital peripheral blood of the above-treated mice was collected and centrifuged at 3000 rpm for 15 min to obtain plasma. The plasma was diluted with a Tris-BSA buffer at 1:200, placed in a PVC microplate, and added with peroxidase-conjugated goat anti-mouse total IgG. Then, a luminescent substrate 3,3′,5,5′-tetramethylbenzidine (TMB) was added for an enzyme-linked immunosorbent assay (ELISA) to evaluate a response of an antibody against factor VIII. HA mice injected with a monoclonal antibody against factor VIII were used as a positive control (Ctrl+). The results are shown in FIG. 13. Factor VIII expressed by F8-BDD-N8 and F8-BDD-299 has a weaker response to the IgG antibody than factor VIII expressed by F8-BDD, and factor VIII expressed by F8-BDD-299 has the weakest response to the IgG antibody.

To sum up, in the present application, the F8-BDD gene is genetically modified and added with sequences of glycosylation sites, and an expression vector is constructed by using the modified lentiviral vector. The expression vector has high transduction and expression efficiency, and the expressed factor VIII is easily secreted outside a cell, has an efficient coagulation function, can correct the bleeding phenotype of HA mice to a certain extent, and has a weak ability to induce an antibody response, which is of great significance for ensuring the effectiveness of gene therapy and lays a basis for relieving HA symptoms faster and achieving more comprehensive and lasting gene therapy.

The applicant has stated that although the detailed method of the present application is described through the examples described above, the present application is not limited to the detailed method described above, which means that the implementation of the present application does not necessarily depend on the detailed method described above. It should be apparent to those skilled in the art that any improvements made to the present application, equivalent substitutions of various raw materials of the product, the addition of adjuvant ingredients, and the selection of specific manners, etc. in the present application all fall within the protection scope and the disclosure scope of the present application.

1. A recombinant coagulation factor VIII, comprising more than 80% of an amino acid sequence as shown in SEQ ID NO: 1 or SEQ ID NO: 2.

2. The recombinant coagulation factor VIII according to claim 1, wherein the recombinant coagulation factor VIII contains a B domain which comprises an amino acid sequence as shown in SEQ ID NO: 8 or SEQ ID NO: 9.

3. A gene encoding the recombinant coagulation factor VIII according to claim 1, wherein the gene encoding the recombinant coagulation factor VIII comprises a nucleic acid sequence as shown in SEQ ID NO: 3 or SEQ ID NO: 4.

4. The gene encoding the recombinant coagulation factor VIII according to claim 3, wherein the gene encoding the recombinant coagulation factor VIII comprises a nucleic acid sequence as shown in SEQ ID NO: 5 or SEQ ID NO: 6.

5. A recombinant expression vector, comprising the gene encoding the recombinant coagulation factor VIII according to claim 3.

6. A recombinant lentivirus containing the gene encoding the recombinant coagulation factor VIII according to claim 3.

7. A recombinant cell containing the gene encoding the recombinant coagulation factor VIII according to claim 3.

8. The recombinant cell according to claim 7, wherein a genome of the recombinant cell is integrated with the gene encoding the recombinant coagulation factor VIII.

9. A method for preparing the recombinant cell according to claim 7, comprising:
introducing the gene encoding the recombinant coagulation factor VIII comprising a nucleic acid sequence as shown in SEQ ID NO: 3 or SEQ ID NO: 4 into a host cell to obtain the recombinant cell.

10. A pharmaceutical composition comprising the recombinant coagulation factor VIII according to claim 1, and
the pharmaceutical composition further comprises any one or a combination of at least two of a pharmaceutically acceptable carrier, excipient or diluent.

11. (canceled)

12. The recombinant expression vector according to claim 5, wherein the recombinant expression vector comprises a viral vector or a plasmid vector containing the gene encoding the recombinant coagulation factor VIII.

13. The recombinant expression vector according to claim 12, wherein the viral vector comprises a lentiviral vector pEGWI.

14. The recombinant expression vector according to claim 13, wherein a 5′ splice donor site GT of the lentiviral vector pEGWI is mutated into CA.

15. The recombinant expression vector according to claim 13, wherein an enhancer in a U3 region of the lentiviral vector pEGWI is deleted, and the U3 region of the lentiviral vector pEGWI contains an insulator.

16. The method according to claim 9, wherein the introduction is carried out by a method which comprises any one of electrical transduction, a viral vector system, a non-viral vector system or direct gene injection; and the host cell comprises a hematopoietic stem cell.

17. A method for treating hemophilia, comprising: administering the recombinant coagulation factor VIII according to claim 1 to a patient in need thereof.
